(19) United States
(12) Patent Application Publication
(10) Pub. No.: US 2017/0100255 A1
(43) Pub. Date: Apr. 13, 2017

(54) INTERVERTEBRAL CAGE FOR LATERAL APPROACH

(71) Applicant: Expanding Orthopedics Inc., Memphis, TN (US)

(72) Inventors: Jaffar Hleihil, Jish (IL); Mark M. Levy, Raanana (IL); Assaf Guy, Allone Abba (IL); Eran Ishay, Tel Aviv (IL)

(73) Assignee: Expanding Orthopedics Inc., Memphis, TN (US)

(21) Appl. No.: 15/155,190

(22) Filed: May 16, 2016

Related U.S. Application Data
(60) Provisional application No. 62/148,215, filed on Apr. 16, 2015.

(51) Int. Cl. A61F 2/44

(52) U.S. Cl. CPC A61F 2/447; A61F 2002/4475; A61F 2220/0091; A61F 2250/0006; A61F 2002/30158

(57) ABSTRACT

An intervertebral cage includes first and second support surfaces arranged for pivoting about a pivot, and one or more lifting cams and an actuating shaft for actuating the cams. Actuation of the cams causes non-parallel movement of one of the first and second support surfaces with respect to the other support surface.

### FIELD OF THE INVENTION

The present invention relates generally to spinal implant devices, and particularly to an intervertebral cage for use through a lateral approach of the spine.

### BACKGROUND OF THE INVENTION

Intervertebral cages for spinal fusion are known devices that help maintain disc space height, assist in the anterior column support of the spine, facilitate the fusion process due to the ability to hold bone graft in place and contribute to the overall alignment of the spine.

Several types of cages are used today through different surgical approaches. One known technique is a lateral approach of the spine for fusion of the thoracic and lumbar spine. Today, standard cages for lateral approach are basically fixed spacers made out of titanium alloys or PEEK (polyether ether ketone) with large chambers for bone graft location. They are usually rectangular in shape to be positioned in the disc space, from one side to the other, after discectomy. The cage crosses the disc space and supports the load basically by direct contact of the cage with the lateral sides of the annular rim of the end plates in addition to the contact at the center of the end plates between them. An example is the ABACUS lateral spacer system from Spine Wave, Inc.

In some cases, the cages may include in its configuration a certain degree of fixed angulation in order to adapt better to the local degree of angulation of the disc space, as in the lordotic shape of lumbar spine. An example is the AVENUE L Lateral Lumbar Cage with VERTEBRIDGE plating from LDR.

Other cages use an expandable feature to increase the height in place and custom fit the disc space. The cage has parallel surfaces that expand. An example is CALIBER-L from Globus Medical.

### SUMMARY OF THE INVENTION

The present invention seeks to provide an improved intervertebral cage for use through a lateral approach of the spine, as is described more in detail hereinbelow. The intervertebral fusion cage can be implanted through the lateral approach in open or minimally invasive surgery, with angular or parallel expansion.

There is thus provided in accordance with an embodiment of the present invention a cage including first and second support surfaces arranged for pivoting about a pivot or flexure, and one or more lifting cams and an actuating shaft for actuating the cams, wherein actuation of the one or more cams causes non-parallel movement of one of the first and second support surfaces with respect to the other support surface.

It is noted that the term “pivot” encompasses an axis or joint about which the surfaces can rotate or flex, including without limitation, a rod, hinge, pin or contiguous surfaces which can flex relative to one another, such as a “live” hinge.

### BRIEF DESCRIPTION OF THE DRAWINGS

The present invention will be understood and appreciated more fully from the following detailed description, taken in conjunction with the drawings in which:

FIGS. 1A and 1B are perspective and side-view illustrations, respectively, of an intervertebral cage, constructed and operative in accordance with a non-limiting embodiment of the invention, in a contracted (non-expanded) orientation.

FIG. 1C is a perspective illustration of the intervertebral cage of FIGS. 1A-1B in an expanded orientation, in accordance with a non-limiting an embodiment of the invention.

FIG. 2 is a see-through perspective illustration of the intervertebral cage, in accordance with a non-limiting an embodiment of the invention.

FIG. 3A is a perspective illustration of the intervertebral cage showing the cage housing, lifting cams and an actuating shaft.

FIG. 3B is a perspective illustration of the assembled intervertebral cage showing the lifting cams and actuating shaft offset from the central longitudinal axis of the cage.

FIGS. 3C, 3D and 3E are perspective, cutaway and further cutaway illustrations, respectively, of the assembled intervertebral cage after expansion, wherein the lifting cams have been actuated to lift one side of the cage.

FIGS. 4A and 4B are top-view and perspective illustrations, respectively, of an intervertebral cage, constructed and operative in accordance with another non-limiting embodiment of the invention, in a contracted (non-expanded) orientation.

FIG. 4C is a perspective illustration of the intervertebral cage of FIGS. 4A-4B in an expanded orientation, in accordance with a non-limiting an embodiment of the invention.

FIG. 5 is a perspective illustration of an intervertebral cage, constructed and operative in accordance with another non-limiting embodiment of the invention, in an expanded orientation.

FIGS. 6A and 6B are side-view illustrations of an intervertebral cage, constructed and operative in accordance with another non-limiting embodiment of the invention, in respective contracted and expanded orientations.

### DETAILED DESCRIPTION OF EMBODIMENTS

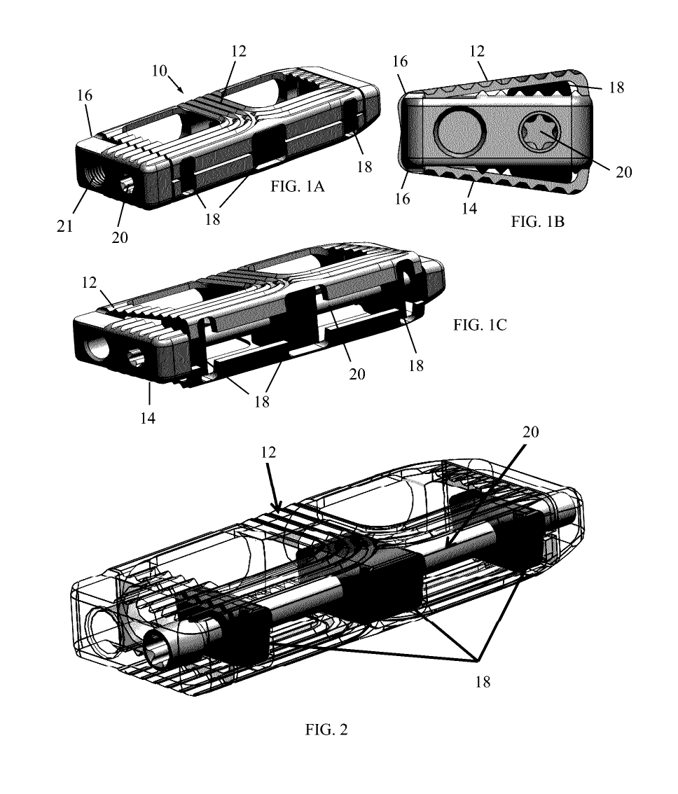

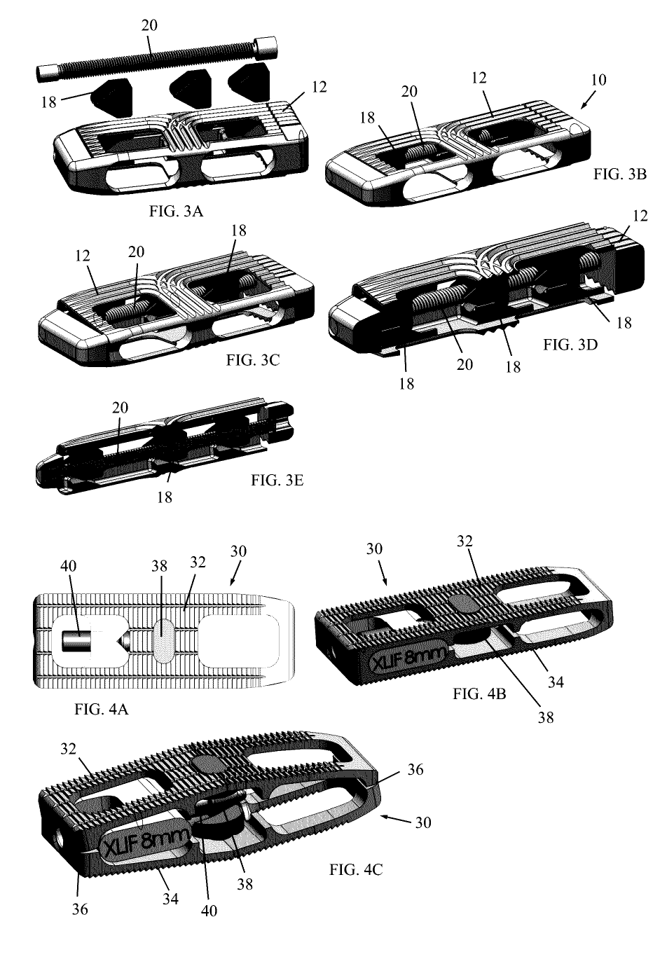

Reference is now made to FIGS. 1A-3E, which illustrate an intervertebral cage 10, constructed and operative in accordance with a non-limiting embodiment of the present invention.

The cage 10 includes first and second support surfaces 12 and 14 (which may be the upper and lower support surfaces), which are arranged for pivoting about a pivot 16 on or near the long (longitudinal) side of cage 10. In other embodiments, the support surfaces can have a built-in angle due to a difference in the thickness of the material in the anterior border of one or both of them, to provide a final enhanced anterior angulation once actuated. The shape of the support surface can be rectangular, arch-shaped, triangular or other shapes. The longitudinal pivot 16 can be a rod which passes through first and second support surfaces 12 and 14. Alternatively, first and second support surfaces 12 and 14 may be flexibly joined at sides of the cage to form a flexible joint which serves as the pivot 16.

The first and second support surfaces 12 and 14 may be serrated for better adhesion or fixation to bone and may have different apertures and/or chambers formed therein for blood circulation, insertion of bone graft materials or bone substitutes, and the like. The first and second support surfaces 12 and 14 may be made of a biocompatible metal like titanium or titanium alloys, a biocompatible polymer like PEEK, a natural material, a synthetic material, a resorbable material or a combination of them. In addition, polymer, metallic or other material cages can be coated with plasma or bone friendly materials including titanium, tantalum, proteins, factors, antibiotics or other drugs. The cage 10 includes one or more lifting cams 18 and an actuating shaft 20 for actuating the cams 18. The cams 18 may be in the shape of wedges, for example. The cams 18 may be mounted on shaft 20 so that turning shaft 20 turns the cams 18 to lift first and/or second support surfaces 12 and 14 (i.e., pivot about pivot 16). The cams 18 can be arranged to lift just one of the support surfaces or both (simultaneously or sequentially). The lifting cams 18 and actuating shaft 20 are offset from the central longitudinal axis of the cage 10.

Non-limiting exemplary dimensions of the cage are: height 8 mm, width 18 mm, length 50 mm, expansion angle 16°, expansion height 12 mm.

The expansion mechanism (cams 18 and turning shaft 20) can be continuous as with a threaded shaft 20 and cams 18 that self-lock at any point of the expansion, or alternatively incrementally, such as with a quarter or half turn of the shaft 20.

Threaded shaft 20 may be supported by bearings and maintained in place by pins, stoppers, snap fasteners, and the like.

All parts of the cage can be cannulated or solid. The shaft is shown as having a TORX 15 head, but it is understood that the invention is not limited to this, and other shafts can be used as well.

The cage is thus easily manipulated and inserted in a lateral approach and then expanded by means of shaft 20 and cams 18. The insertion can be done at any side of the spine, right or left, by simply turning the device. The expansion can be done through part or all of the available threaded range and can be stopped at any point, resulting in an inherent locking of the device at that given position. For example, the expansion may be stopped by the cams or wedges reaching the end of travel on the corresponding inclined surface or may be stopped by abutting against a stopper provided in the cage.

Besides the access orifice of shaft 20, there is another aperture 21 used for holding the device with an insertion tool (or delivery instrument). The delivery instrument is reversibly attached to the device, and in some embodiments is hollow to allow the insertion of bone graft or other substances to the inner chambers of the cage and the disc space. In another embodiment, the same aperture for attachment of the delivery instrument can also be used for the actuator shaft 20. The opposite end of the device, which leads the introduction of the device, may have a more rounded shape, chamfered or bullet shaped, to allow a smooth introduction of the device into the disc space.

Reference is now made to FIGS. 4A-4C, which illustrate an intervertebral cage 30, constructed and operative in accordance with a non-limiting embodiment of the present invention.

The cage 30 includes first and second support surfaces 32 and 34 (which may be the upper and lower support surfaces). Each support surface has an end arranged for pivoting about a pivot 36 on or near the short (end) side of cage 30. The pivot 36 can be a rod which passes through the end portion of the support surface. Alternatively, the ends of first and second support surfaces 12 and 14 may be flexibly joined at ends of the cage to form a flexible joint which serves as the pivot 36.

The cage 30 includes one or more lifting cams 38 and an actuating shaft 40 for actuating the cams 38. The cams 38 may be in the shape of wedges, for example. The cams 38 may be mounted on shaft 40 so that turning shaft 40 turns the cams 38 to lift first and/or second support surfaces 32 and 34 (i.e., pivot about pivots 36), for example, by the action of the wedges or cams sliding on an inclined surface or surfaces. The cams 38 can be arranged to lift just one of the support surfaces or both (simultaneously or sequentially). In the illustrated embodiment, there is one central cam 38 and shaft 40, which are positioned on the central longitudinal axis of the cage 30 (however, alternatively they can be offset as in the first embodiment). Actuating cam 38 raises the central portion of cage 30 to obtain variable angulation (as opposed to the prior art fixed angulation) for properly adapting to the local degree of angulation and shape of the disc space, as in the thoracic or lumbar spine and increasing the disc space height. The angulated expansion can be facing the anterior border of the cage or the center of the cage.

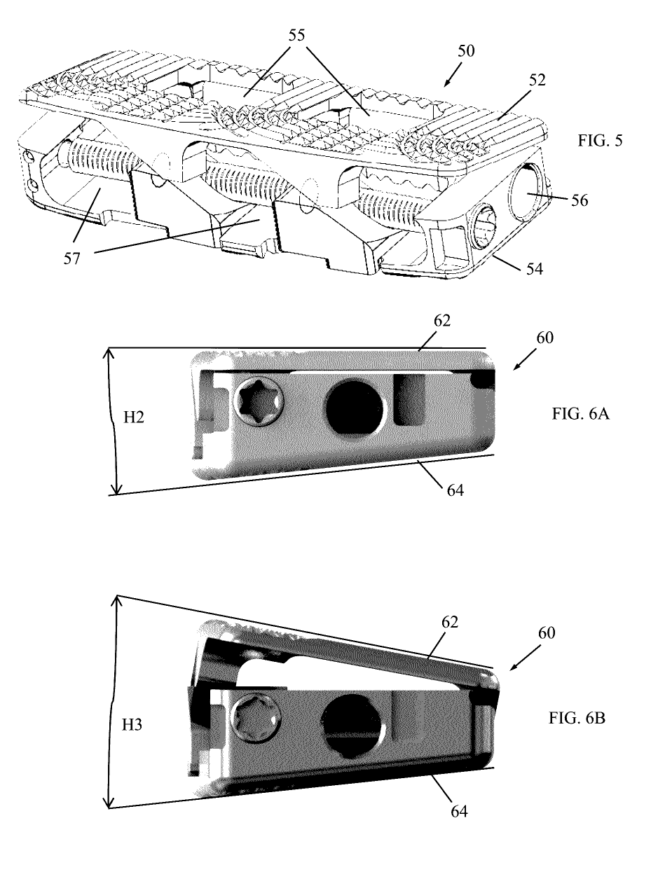

Reference is now made to FIG. 5, which illustrates an intervertebral cage 50, constructed and operative in accordance with another non-limiting embodiment of the invention, in an expanded orientation.

The cage 50 includes first and second support surfaces 52 and 54 (which may be the upper and lower support surfaces), which are arranged for pivoting about a pivot 56 at or near (e.g., anterior or posterior to) the long (longitudinal) side of cage 50. It is noted that in this embodiment, the entire first (upper) support surface 52 is expandable; there is no rim or border that does not expand (which was the case in the illustrated embodiment of FIGS. 1A-1C).

Cage 50 may be formed with windows or cutouts 55 in first and/or second support surfaces 52 and 54, as well as additional windows or cutouts 57 in the lateral walls of cage 50. The first and/or second support surfaces 52 and 54 may be formed with sharp or trapezoid edges or any other rough surface to better grip the bone surfaces in the direction of expansion and allow bone ingrowth. In addition, in this and any other embodiments, the surfaces facing the endplates can be coated, processed with laser etching, plasma spray, blast media, porous coating, photochemical etching or other surface processing methods.

Cage 50 or any of the other cages of the invention may be optionally formed with a bullet or tapering nose for easy insertion.

Cage 50 or any of the other cages of the invention may be constructed, without limitation, of medically safe metals (e.g., stainless steel alloys, titanium or titanium alloys), plastics or elastomeric materials (e.g., different kinds of polymers, natural, synthetic and resorbable) and others.

Instead of using cams, cage 50 or any of the other cages of the invention may be expanded, without limitation, by using a ratchet mechanism, by using electrical, hydraulic or pneumatic actuators, using shape memory materials, or by using a balloon.

Reference is now made to FIGS. 6A and 6B, which illustrate an intervertebral cage 60, constructed and operative in accordance with another non-limiting embodiment of the invention, in respective contracted and expanded orientations. Cage 60, without limitation, may be particularly useful correcting lack of lordosis.

Cage 60 includes first and second support surfaces 62 and 64 (which may be the upper and lower support surfaces), which are arranged for pivoting about a pivot 66 on or near the long (longitudinal) side of cage 60. In this embodiment, the support surfaces 62 and 64 can have a built-in angle due to a difference in the thickness of the material in the anterior border of one or both of them, to provide a final enhanced anterior angulation once actuated. Accordingly, lateral cage 60 has a built-in angulation which can be expanded to a greater angle. For example, without limitation, in the pre-expanded orientation (FIG. 6A), the angle between first and second support surfaces 62 and 64 may be 6° (fixed lordosis angle) and the height H2 between first and second support surfaces 62 and 64 may be 10 mm. Without limitation, in the expanded orientation (FIG. 6B), the angle between first and second support surfaces 62 and 64 may be 10-30° (total lordosis angle) and the height H3 between first and second support surfaces 62 and 64 may be 14 mm or greater.

Any of the cages of the invention may be expanded with a threaded tool or other tool mounted or assembled on the insertion tool, as opposed to a threaded fastener and the like which are included in the cage.

The orifices of any of the cages of the invention may be of any size and position and may be used for inserting bone graft or other materials and/or tools, such as insertion tools. For example, the pivot side of the cage can be formed with orifices at any point or side of the pivot. The cage can be provided with a threaded or other connection at a surface opposite or alongside the insertion face of the cage for removal of the cage.

What is claimed is:

1. A device comprising:
an intervertebral cage comprising first and second support surfaces arranged for pivoting about a pivot; and
one or more lifting cams and an actuating shaft for actuating the cams, wherein actuation of said one or more cams causes non-parallel movement of one of said first and second support surfaces with respect to the other support surface.

2. The device according to claim 1, wherein the pivot is on or near a longitudinal side of said cage.

3. The device according to claim 1, wherein the pivot is on or near a non-longitudinal end of said cage.

4. The device according to claim 1, wherein said lifting cams and said actuating shaft are offset from a central longitudinal axis of the cage.

5. The device according to claim 1, wherein said lifting cams and said actuating shaft are located on a central longitudinal axis of the cage.

6. The device according to claim 1, wherein said lifting cams raise a central portion of said cage to obtain variable angulation of one of said first and second support surfaces.

7. The device according to claim 1, wherein said first and second support surfaces have apertures and/or chambers formed therein.

8. The device according to claim 1, wherein said cage is formed with an opening for receiving therein a tool.

9. The device according to claim 1, wherein said cage is formed with an opening for introducing therethrough a material.
